(19) United States
(12) Patent Application Publication
(10) Pub. No.: US 2014/0243533 A1
(43) Pub. Date: Aug. 28, 2014

(54) METHOD FOR PRODUCTION OF F-18 LABELED A-BETA LIGANDS

(71) Applicant: PIRAMAL IMAGING SA, Matran (CH)

(72) Inventors: Franz Meier, Berlin (DE); Keith Graham, Berlin (DE)

(73) Assignee: PIRAMAL IMAGING SA, Matran (CH)

(21) Appl. No.: 14/352,007

(22) PCT Filed: Oct. 18, 2012
(86) PCT No.: PCT/EP2012/070669

§ 371 Date: Apr. 15, 2014

(30) Foreign Application Priority Data
Oct. 19, 2011 (EP) 11075236.7

(51) Int. Cl. C07C 213/08; C07D 213/64

(52) U.S. Cl. CPC C07C 213/08; C07D 213/64
USPC 546/300; 564/412

(57) ABSTRACT

This invention relates to improved methods, which provide access to [F-18]fluoropegylated (aryl/heteroaryl vinyl)-phenyl methyl amine derivatives.

### FIELD OF INVENTION

This invention relates to an improved method, which provides access to [F-18]fluoropegylated (aryl/heteroaryl vinyl)-phenyl methyl amine derivatives.

### BACKGROUND

Alzheimer's Disease (AD) is a progressive neurodegenerative disorder marked by loss of memory, cognition, and behavioral stability. AD is defined pathologically by extracellular senile plaques comprised of fibrillar deposits of the beta-amyloid peptide (Aβ) and neurofibrillary tangles comprised of paired helical filaments of hyperphosphorylated tau. The 39-43 amino acids comprising Aβ peptides are derived from the larger amyloid precursor protein (APP). In the amyloidogenic pathway, Aβ peptides are cleaved from APP by the sequential proteolysis by beta- and gamma-secretases. Aβ peptides are released as soluble proteins and are detected at low level in the cerebrospinal fluid (CSF) in normal aging brain. During the progress of AD the Aβ peptides aggregate and form amyloid deposits in the parenchyma and vasculature of the brain, which can be detected post mortem as diffuse and senile plaques and vascular amyloid during histological examination (for a recent review see: Blennow et al. Lancet. 2006 Jul. 29; 368(9533):387-403).

Alzheimer's disease (AD) is becoming a great health and social economical problem all over the world. There are great efforts to develop techniques and methods for the early detection and effective treatment of the disease. Currently, diagnosis of AD in an academic memory-disorders clinic setting is approximately 85-90% accurate (Petrella J R et al. Radiology. 2003 226:315-36). It is based on the exclusion of a variety of diseases causing similar symptoms and the careful neurological and psychiatric examination, as well as neuropsychological testing.

Molecular imaging has the potential to detect disease progression or therapeutic effectiveness earlier than most conventional methods in the fields of neurology, oncology and cardiology. Among the several promising molecular imaging technologies, such as optical imaging, MRI, SPECT and PET, PET is of particular interest for drug development because of its high sensitivity and ability to provide quantitative and kinetic data.

For example positron emitting isotopes include e.g. carbon, iodine, nitrogen and oxygen. These isotopes can replace their non-radioactive counterparts in target compounds to produce PET tracers that have similar biological properties. Among these isotopes F-18 is a preferred labeling isotope due to its half life of 110 min, which permits the preparation of diagnostic tracers and subsequent study of biochemical processes. In addition, its low β+ energy (634 keV) is also advantageous.

Post-mortem histological examination of the brain is still the only definite diagnosis of Alzheimer's disease. Thus, the in vivo detection of one pathological feature of the disease—the amyloid aggregate deposition in the brain—is thought to have a strong impact on the early detection of AD and differentiating it from other forms of dementia. Additionally, most disease modifying therapies which are in development are aiming at lowering of the amyloid load in the brain. Thus, imaging the amyloid load in the brain may provide an essential tool for patient stratification and treatment monitoring (for a recent review see: Nordberg. Eur J Nucl Med Mol Imaging. 2008 March; 35 Suppl 1:S46-50).

In addition, amyloid deposits are also known to play a role in amyloidoses, in which amyloid proteins (e.g. tau) are abnormally deposited in different organs and/or tissues, causing disease. For a recent review see Chiti et al. Annu Rev Biochem. 2006; 75:333-66.

Fluoropegylated (aryl/heteroaryl vinyl)-phenyl methyl amines such as 4-[(E)-2-(4-{2-[2-(2-fluoroethoxy)ethoxy]ethoxy}phenyl)vinyl]-N-methylaniline and 4-[(E)-2-(6-{2-[2-(2-fluoroethoxy)ethoxy]ethoxy}pyridin-3-yl)vinyl]-N-methylaniline have been labeled with F-18 fluoride and are covered by patent applications WO2006066104, WO2007126733 and members of the corresponding patent families.

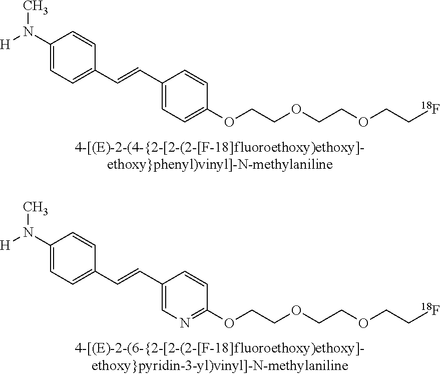 [SMILES: CNc1ccc(/C=C/c2ccc(OCCOCCOCC[18F])cc2)cc1 ; CNc1ccc(/C=C/c2ccc(OCCOCCOCC[18F])nc2)cc1]

The usefulness of these radiotracers for the detection of Aβ plaques have been reported in the literature (W. Zhang et al., Nuclear Medicine and Biology 32 (2005) 799-809; C. Rowe et al., Lancet Neurology 7 (2008) 1-7; S. R. Choi et al., The Journal of Nuclear Medicine 50 (2009) 1887-1894).

To not limit the use of such F-18 labeled diagnostics, processes are needed, that allow a robust and safe manufacturing of the F-18 labeled tracers. Additionally, such processes should provide high yield of the overall synthesis to allow the production of quantities of the diagnostic to supply the radiotracer, despite of the half life of 110 min, to facilities without cyclotron or radiopharmaceutical production facility.

Syntheses of F-18 labeled fluoropegylated (aryl/heteroaryl vinyl)-phenyl methyl amines have been described before. For most of these radiosyntheses a F-18 eluent mixture typically comprises kryptofix 2.2.2, potassium carbonate, acetonitrile and water.

### 4-[(E)-2-(4-{2-[2-(2-[F-18]fluoroethoxy)ethoxy]ethoxy}phenyl)vinyl]-N-methylaniline

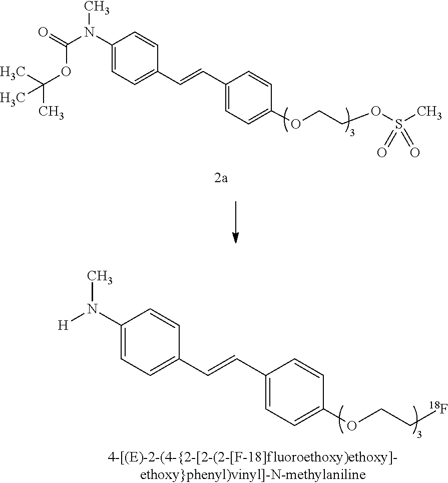

### a) W. Zhang et al., Nuclear Medicine and Biology 32 (2005) 799-809

4 mg precursor 2a (2-[2-(2-{4-[(E)-2-{4-[(tert-butoxycarbonyl)(methyl)amino]-phenyl}vinyl]phenoxy}ethoxy)ethoxy]ethyl methanesulfonate) in 0.2 mL DMSO were reacted with [F-18]fluoride/kryptofix/potassium carbonate complex. The intermediate was deprotected with HCl and neutralized with NaOH. The mixture was extracted with ethyl acetate. The solvent was dried and evaporated. The residue was dissolved in acetonitrile and purified by semi-preparative HPLC (acetonitrile/5 mM dimethylglutarate buffer pH 7 9/1). 20% (decay corrected), 11% (not corrected for decay) 4-[(E)-2-(4-{2-[2-(2-[F-18]fluoroethoxy)ethoxy]ethoxy}phenyl)vinyl]-N-methylaniline were obtained within 90 min. An additional re-Formulation, necessary to obtain a solution suitable for injection into human is not described.

### b) WO2006066104

4 mg precursor 2a (2-[2-(2-{4-[(E)-2-{4-[(tert-butoxycarbonyl)(methyl)amino]-phenyl}vinyl]phenoxy}ethoxy)ethoxy]ethyl methanesulfonate) in 0.2 mL DMSO were reacted with [F-18]fluoride/kryptofix/potassium carbonate complex. The intermediate was deprotected with HCl and neutralized with NaOH. The mixture was extracted with ethyl acetate. The solvent was dried and evaporated, the residue was dissolved in acetonitrile and purified by semi-preparative HPLC. 30% (decay corrected), 17% (not corrected for decay) 4-[(E)-2-(4-{2-[2-(2-[F-18]fluoroethoxy)ethoxy]ethoxy}phenyl)vinyl]-N-methylaniline were obtained in 90 min. An additional re-Formulation, necessary to obtain a solution suitable for injection into human is not described.

### c) H. Wang et al., Nuclear Medicine and Biology 38 (2011) 121-127

5 mg (9.33 μmol) precursor 2a (2-[2-(2-{4-[(E)-2-{4-[(tert-butoxycarbonyl)(methyl)amino]-phenyl}vinyl]phenoxy}ethoxy)ethoxy]ethyl methanesulfonate) in 0.5 mL DMSO were reacted with [F-18]fluoride/kryptofix/potassium carbonate complex. The intermediate was deprotected with HCl and neutralized with NaOH. The crude product was diluted with acetonitrile/0.1M ammonium formate (6/4) and purified by semi-preparative HPLC. The product fraction was collected, diluted with water, passed through a C18 cartridge and eluted with ethanol, yielding 17% (not corrected for decay) 4-[(E)-2-(4-{2-[2-(2-[F-18]fluoroethoxy)ethoxy]ethoxy}phenyl)vinyl]-N-methylaniline within 50 min.
In the same paper, the conversion of an unprotected mesylate precursor (is described:
5 mg (11.48 μmol) unprotected mesylate precursor (2-{2-[2-(4-{(E)-2-[4-(methylamino)phenyl]vinyl}phenoxy)ethoxy]-ethoxy}ethyl 4-methanesulfonate) in 0.5 mL DMSO were reacted with [F-18]fluoride/kryptofix/potassium carbonate complex. The crude product was diluted with acetonitrile/0.1M ammonium formate (6/4) and purified by semi-preparative HPLC. The product fraction was collected, diluted with water, passed through a C18 cartridge and eluted with ethanol, yielding 23% (not corrected for decay) 4-[(E)-2-(4-{2-[2-(2[F-18]fluoroethoxy) ethoxy]ethoxy}phenyl)vinyl]-N-methylaniline within 30 min.
A process wherein the radiotracer was purified by SPE (without HPLC) only, was found to afford a product with acceptable radiochemical purity (>95%), however, the chemical purity was too low, e.g. side products derived from the excess of precursor could not be removed.

### d) US20100113763

2a (2-[2-(2-{4-[(E)-2-{4-[(tert-butoxycarbonyl)(methyl)amino]phenyl}vinyl]-phenoxy}ethoxy)ethoxy]ethyl methanesulfonate) was reacted with [F-18]fluoride reagent in a mixture of tert-alcohol and acetonitrile. After fluorination, the solvent was evaporated and a mixture of HCl and acetonitrile was added. After deprotection (heating at 100-120° C.), the crude product mixture was purified by HPLC (C18, 60% acetonitrile, 40% 0.1M ammonium formate). An additional re-Formulation, necessary to obtain a solution suitable for injection into human is not described.

### 4-[(E)-2-(6-{2-[2-(2-[F-18]fluoroethoxy)ethoxy]ethoxy}pyridin-3-yl)vinyl]-N-methylaniline

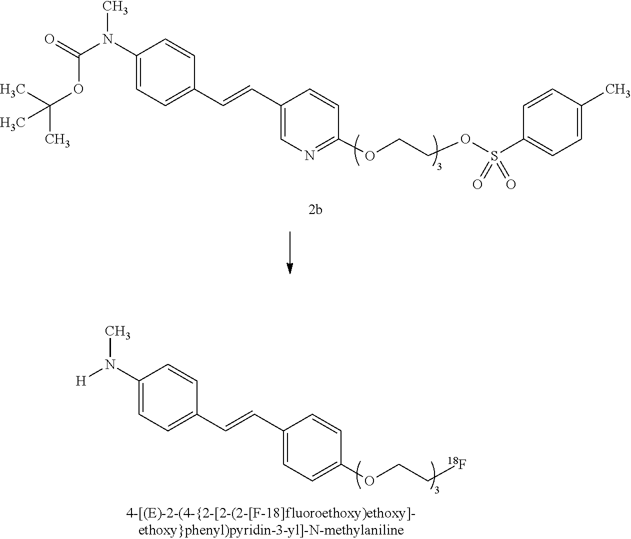

### a) S. R. Choi et al., The Journal of Nuclear Medicine 50 (2009) 1887-1894.

1 mg precursor 2b (2-{2-[2-({5-[(E)-2-{4-[(tert-butoxycarbonyl)(methyl)amino]-phenyl}vinyl]pyridin-2-yl}oxy)ethoxy]ethoxy}ethyl 4-methylbenzenesulfonate) in 1 mL DMSO was reacted with [F-18]fluoride/kryptofix/potassium carbonate complex. The intermediate was deprotected with HCl and neutralized with NaOH. DMSO and inorganic components were removed by solid-phase-extraction on SepPak light C18 cartridge (Waters). The crude product was purified by semi-preparative HPLC (55% acetonitrile, 45% 20 mM NH₄OAc+0.5% w/v sodium ascorbate). The product fraction was diluted with water and passed through a SepPak light C18 cartridge. The radiotracer was eluted with ethanol. The yield for 4-[(E)-2-(6-{2-[2-(2-[F-18]fluoroethoxy)ethoxy]ethoxy}pyridin-3-yl)vinyl]-N-methylaniline was 10-30% (decay corrected).

### b) WO2010078370

1.5 mg (2.45 μmol) precursor 2b (2-{2-[2-({5-[(E)-2-{4-[(tert-butoxycarbonyl)(methyl)amino]-phenyl}vinyl]pyridin-2-yl}oxy)ethoxy]ethoxy}ethyl 4-methylbenzenesulfonate) in 2 mL DMSO was reacted with [F-18]fluoride/kryptofix/potassium carbonate complex. The intermediate was deprotected with HCl and diluted with 1% NaOH solution for neutralization. The mixture was loaded onto a reverse phase cartridge. The cartridge was washed with water (containing 5% w/v sodium ascorbate). The crude product was eluted with acetonitrile into a reservoir containing water+5% w/v sodium ascorbate and HPLC solvent. After purification by semi-preparative HPLC, the product fraction was collected into a reservoir containing water+0.5% w/v sodium ascorbate. The solution was passed trough a C18 cartridge, the cartridge was washed with water (containing 0.5% w/v sodium ascorbate and the final product was eluted with ethanol into a vial containing 0.9% sodium chloride solution with 0.5% w/v sodium ascorbate.

### c) Y. Liu et al., Nuclear Medicine and Biology 37 (2010) 917-925

1 mg (1.63 μmol) precursor 2b (2-{2-[2-({5-[(E)-2-{4-[(tert-butoxycarbonyl)(methyl)amino]-phenyl}vinyl]pyridin-2-yl}oxy)ethoxy]ethoxy}ethyl 4-methylbenzenesulfonate) in 1 mL DMSO was reacted with [F-18]fluoride/kryptofix/potassium carbonate complex. The intermediate was deprotected with HCl and diluted with 1% NaOH solution. The mixture was loaded onto a Oasis HLB cartridge. The cartridge was washed with water, dried under a flow of argon and the product was eluted with ethanol into a vial containing a saline solution. Although, radiochemical impurities were removed by this procedure, non-radioactive by-products derived from hydrolysis of the excess of precursor, remained in the final product solution.
The yield for 4-[(E)-2-(6-{2-[2-(2-[F-18]fluoroethoxy)ethoxy]ethoxy}pyridin-3-yl)vinyl]-N-methylaniline was 34% (non-decay corrected) within 50 min at a radioactive level from 10-100 mCi (370-3700 MBq) of [F-18]fluoride.

### d) L. Silva et al., Abstract/Poster EANM 2010

An IBA Synthera platform was adapted for the synthesis of 4-[(E)-2-(6-{2-[2-(2-[F-18]fluoroethoxy)ethoxy]ethoxy}pyridin-3-yl)vinyl]-N-methylaniline. Additionally, a semi-preparative HPLC system and a further Synthera module for re-Formulation was integrated.
   e) G. Casale et al. World Journal of Nuclear Medicine, 9 S1 (2010), S-174 (Abstract of 10^{th} Congress of WFNMB, Cape Town, South Africa, 18-23 Sep. 2010)
The manufacturing of 4-[(E)-2-(6-{2-[2-(2-[F-18]fluoroethoxy)ethoxy]ethoxy}pyridin-3-yl)vinyl]-N-methylaniline have been accomplished by use of an IBA Synthera synthesis module, combined with an HPLC semi preparative purification system and an additional module for Formulation (dilution of HPLC fraction, trapping on a C18 cartridge, washing and elution with ethanol).

The general setup of the manufacturing process for F-18 labeled fluoropegylated (aryl/heteroaryl vinyl)-phenyl methyl amines as previously described is illustrated in FIG. 4. The manufacturing process can be divided into three major parts:
A) Synthesis
B) Purification by HPLC
C) Formulation

During the first manufacturing step [F-18]fluoride is eluted from a ion exchange cartridge (e.g. QMA) usually using an eluent mixture comprising cryptand (e.g. kryptofix 2.2.2), a base (e.g. potassium carbonate), acetonitrile and water. The manufacturing steps are:
i) drying of [F-18]fluoride
ii) radiolabeling of the precursor molecule
iii) deprotection.

These steps outlined above are performed on the part A of the synthesis device (FIG. 4). The crude product mixture is transferred to the second part B for purification by HPLC (on reversed phase silica gel using acetonitrile/buffer eluent) with the product peak being collected. To obtain the radiotracer in a Formulation, suitable for injection into human, the solvent (acetonitrile) present in the collected HPLC product fraction needs to be removed and exchanged by a composition that is appropriate for the manufacturing of a medicament. To avoid this, HPLC solvents can be used that are compatible with injectable formuations, such as ethanol/water based solvents.

Typically (and described in the references above), the product fraction is diluted with water (vessel “8”, FIG. 4, part C) and then passed through a reversed phase cartridge (“11”, FIG. 4, part C). The cartridge is washed with a aqueous solution from one of the reservoirs “9” (FIG. 4, part C) and finally eluted from the cartridge with an ethanolic solution (or ethanol) from another of the reservoirs “9” into the product vial, that can optionally comprise of further parts and excipients to give a final Formulation. It is obvious to those skilled in the art, that the illustration in FIG. 4 is a simplification of process and equipment and that further parts such as valves, vials, tubing etc. can be part of such process or equipment.

A “GMP compliant” manufacturing process for 4-[(E)-2-(6-{2-[2-(2-[F-18]fluoroethoxy)ethoxy]-ethoxy}pyridin-3-yl)vinyl]-N-methylaniline is disclosed in WO2010078370 and C.-H. Yao et al., Applied Radiation and Isotopes 68 (2010) 2293-2297. To prevent the decomposition of 4-[(E)-2-(6-{2-[2-(2-[F-18]fluoroethoxy)ethoxy]-ethoxy}pyridin-3-yl)vinyl]-N-methylaniline, sodium ascorbate was added to the HPLC solvent (45% acetonitrile, 55% 20 mM ammoniumacetate containing 0.5% (w/v) sodium ascorbate) and the final Formulation (0.5% (w/v) sodium ascorbate). The process afforded up to 18.5 GBq (25.4±7.7%, decay corrected) 4-[(E)-2-(6-{2-[2-(2-[F-18]fluoroethoxy)ethoxy]-ethoxy}pyridin-3-yl)vinyl]-N-methylaniline. The radiochemical purity was 95.3±2.2%.

Although, cartridge based purification processes have been investigated, an optimum of product quality regarding radiochemical purity and separation from (non-radioactive) by-products have been demonstrated and proofed only for HPLC purification.

Here, a new eluation method for [F-18]-fluoride in the first manufacturing step is provided.

To take full advantage of the new purification method it turned out to be benefical to start with a crude product which can be purified easily by HPLC whereby the mobile phase comprises of an ethanol/buffer mixture. Typically [F-18]fluoride is trapped on a anion exchange resin (e.g. QMA cartridge or SPE) and then eluted from this anion exchange resin with a basic solution consisting of base, acetonitrile and water and optionally a cryptand. The most commonly used solution for elution of [F-18]fluoride trapped on a anion exchange resin comprise of varying amounts of kryptofix 2.2.2. (cryptand), potassium carbonate (base), acetonitrile and water (conventional eluent mixture). However, in particular if older batches of this conventional eluent mixture or conventional eluent batches from accelerated stability studies are used, in case of the synthesis of 4-[(E)-2-(4-{2-[2-(2-fluoroethoxy)ethoxy]ethoxy}phenyl)vinyl]-N-methylaniline the crude product contains major amounts of a chemical impurity (Example 3 and 4, Table 4 and 5) which also can be found in minor amounts even in the purified product solution (Example 7, Table 9). The chemical impurity has very similar chromatographic properties as F-18 compound and has been identified as 2-{2-[2-(4-{(E)-2-[4-(methylamino)phenyl]vinyl}phenoxy)ethoxy]-ethoxy}ethyl acetate; this compound is formed by the substitution of the mesylate leaving group by acetate present in the eluting solution: This undesired substitution will compete with the [F-18]fluoride substitution affecting both the yield of the desired PET and additionally give more chemical impurities that require removal by HPLC. Surprisingly this acetate substitution and its corresponding impurity can be almost completely avoided if the conventional F-18 eluent mixture is replaced by a solution of kryptofix 2.2.2., potassium carbonate, a low boiling alcohol and water. A. Svadberg et al reported recently on the XII Turku PET Symposium (28-31 May 2011, Turku, Finland) and at the Society of Nuclear Medicine Annual Meeting (4-8 Jun. 2011, San Antonio, USA) about the chemical instability of the conventional eluent mixture. Upon storage of the eluent acetamide and ammonium acetate are formed by alkaline hydrolysis of acetonitrile. The higher the acetate content of the eluent the lower is the radiochemical yield of the F-18 compound and the higher is the content of the formed acetate impurity. So the use of the eluent solution comprising kryptofix 2.2.2, potassium carbonate, a low boiling alcohol and water combines the double advantage giving rise to a cleaner product and a constant good yield which is independent of the storage conditions of the F-18 eluent.

A further major advantage of the new method described herein, is the reliably high radiochemical purity of the F-18 labeled fluoropegylated (aryl/heteroaryl vinyl)-phenyl methyl amines synthesized by the new method. Especially at higher radioactivity levels (>20 GBq) radiochemical purities >95% were achieved.

### SUMMARY OF THE INVENTION

The present invention provides a method for production of a radiolabeled compound of Formula I and suitable salts of an inorganic or organic acid thereof, hydrates, complexes, esters, amides, solvates and prodrugs thereof.
   The method comprises the steps of:
      Elution of F-18 trapped on a cartridge using a eluent solution comprising kryptofix 2.2.2, potassium carbonate, an alcohol having a boiling point below 100° C. and water and evaporation of the solvent
      Radiofluorination of compound of Formula II
      Optionally, cleavage of a protecting group
      Purification and Formulation of compound of Formula I by HPLC. Preferably, ethanolic solutions with aqueous buffer solutions containing ascorbic acid that can be part of an injectable Formulation are used as HPLC eluents.

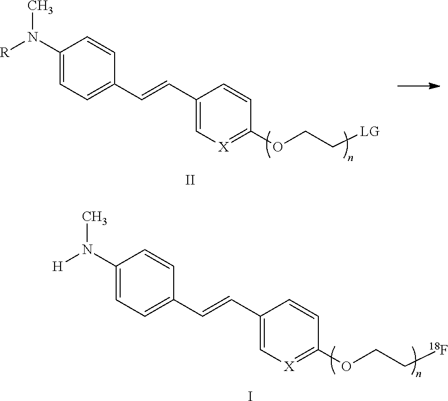

One way to carry out the method provided by the present invention is schematically illustrated in FIG. 5. Radiofluorination of compound of Formula II and optionally, the cleavage of a protecting group are performed on the left-hand part of the equipment (FIG. 5, part A). The purification of compound of Formula I is performed in a way, that the product fraction obtained by HPLC (FIG. 5, part B) can be directly transferred into the product vial, wherein the product vial optionally contains further pharmaceutically acceptable carriers, diluents, adjuvant or excipients. A further part of process and equipment as illustrated in FIG. 4 (Part C) is not longer required by the Method of the present invention.
The present invention also provides compositions comprising a radiolabeled compound of Formula I or suitable salts of an inorganic or organic acid thereof, hydrates, complexes, esters, amides, solvates and prodrugs thereof and optionally a pharmaceutically acceptable carrier, diluent, adjuvant or excipient.
The present invention also provides a Kit for preparing a radiopharmaceutical preparation by the herein described process, said Kit comprising a sealed vial containing a predetermined quantity of the compound of Formula II.

### DESCRIPTION OF THE INVENTION

In a first aspect the present invention is directed to a Method for producing compound of Formula I

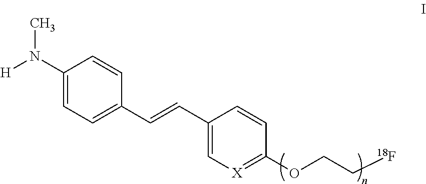

comprising the steps of:
Step 1: Preparation a fluorinating agent by eluting [18F]fluoride trapped on a cartridge using a mixture of cryptand, base, water and an alcohol having a boiling point below 100° C. followed by evaporation of the solvents.
Step 2: Radiolabeling compound of Formula II with the F-18 fluorinating agent, to obtain compound of Formula I, if R=H or to obtain compound of Formula III, if R=PG

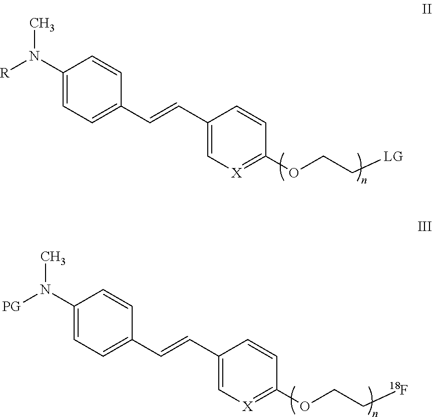

Step 3: Optionally, if R=PG, cleavage of the protecting group PG to obtain compound of Formula I
Step 4: Purification and optionally formulation of compound of Formula I
   wherein:
   n=1-6, preferably 2-4, more preferably 3.

X is selected from the group comprising

### a) CH,

### b) N.

In one preferred embodiment, X═CH.

In another preferred embodiment, X═N.

R is selected from the group comprising

### a) H,

### b) PG.

PG is an “Amine-protecting group”.

In a preferred embodiment, PG is selected from the group comprising:

### a) Boc,

### b) Trityl and

### c) 4-Methoxytrityl.

In a more preferred embodiment, R is H.

In another more preferred embodiment, R is Boc.

LG is a Leaving group.

In a preferred embodiment, LG is selected from the group comprising:

### a) Halogen and

### b) Sulfonyloxy.

Halogen is chloro, bromo or iodo. Preferably, Halogen is bromo or chloro.

In a preferred embodiment Sulfonyloxy is selected from the group consisting of Methanesulfonyloxy, p-Toluenesulfonyloxy, Trifluormethylsulfonyloxy, 4-Cyanophenylsulfonyloxy, 4-Bromophenylsulfonyloxy, 4-Nitrophenylsulfonyloxy, 2-Nitrophenylsulfonyloxy, 4-Isopropyl-phenylsulfonyloxy, 2,4,6-Triisopropyl-phenylsulfonyloxy, 2,4,6-Trimethylphenylsulfonyloxy, 4-tert-Butyl-phenylsulfonyloxy, 4-Adamantylphenylsulfonyloxy and 4-Methoxyphenylsulfonyloxy.

In a more preferred embodiment, Sulfonyloxy is selected from the group comprising:

### a) Methanesulfonyloxy,

b) p-Toluenesulfonyloxy,

### c) (4-Nitrophenyl)sulfonyloxy,

### d) (4-Bromophenyl)sulfonyloxy.

In a even more preferred embodiment LG is Methanesulfonyloxy.

In another even more preferred embodiment LG is p-Toluenesulfonyloxy.

A preferred compound of Formula I is:

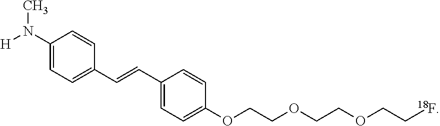

### 4[(E)-2-(4-{2-[2-(2[F-18]fluoroethoxy)ethoxy]ethoxy}phenyl)vinyl]-N-methylaniline

Another preferred compound of Formula I is:

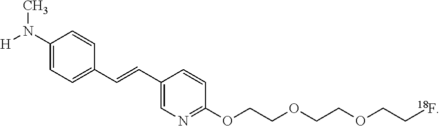

### 4-[(E)-2-(6-{2-[2-(2-[F-18]fluoroethoxy)ethoxy]ethoxy}pyridin-3-yl)vinyl]-N-methylaniline

A preferred compound of Formula II is:

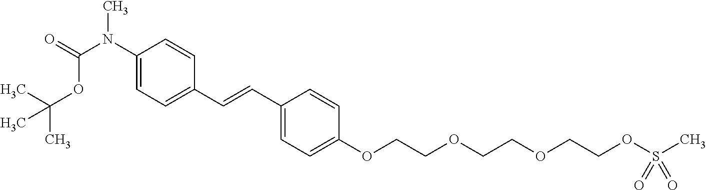 [SMILES: CN(C(=O)OC(C)(C)C)c1ccc(/C=C/c2ccc(OCCOCCOCCOS(C)(=O)=O)cc2)cc1]

### 2-[2-(2-{4-[(E)-2-{4-[(tert-butoxycarbonyl)(methyl)amino]phenyl}vinyl]phenoxy}-ethoxy)ethoxy]ethyl methanesulfonate

Another preferred compound of Formula II is:

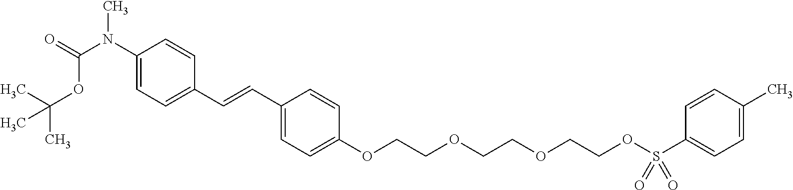 [SMILES: Cc1ccc(S(=O)(=O)OCCOCCOCCOc2ccc(/C=C/c3ccc(N(C)C(=O)OC(C)(C)C)cc3)cc2)cc1]

### 2-[2-(2-{4-[(E)-2-{4-[(tert-butoxycarbonyl)(methyl)amino]phenyl}vinyl]phenoxy}-ethoxy)ethoxy]ethyl 4-methylbenzenesulfonate

Another preferred compound of Formula II is:

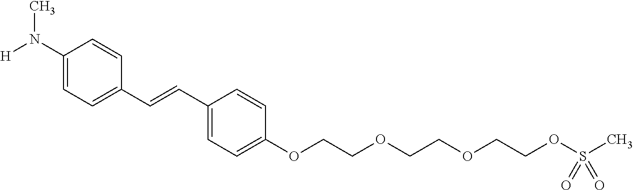 [SMILES: CNc1ccc(/C=C/c2ccc(OCCOCCOCCOS(C)(=O)=O)cc2)cc1]

### 2-{2-[2-(4-{(E)-2-[4-(methylamino)phenyl]vinyl}phenoxy)ethoxy]ethoxy}ethyl 4-methylbenzenesulfonate

Another preferred compound of Formula II is:

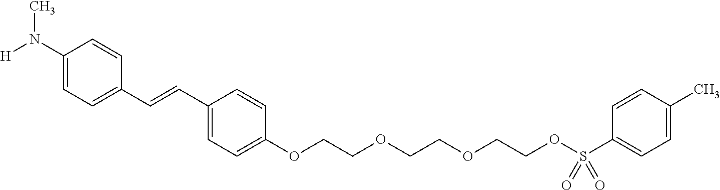 [SMILES: CNc1ccc(/C=C/c2ccc(OCCOCCOCCOS(=O)(=O)c3ccc(C)cc3)cc2)cc1]

### 2-{2-[2-(4-{(E)-2-[4-(methylamino)phenyl]vinyl}phenoxy)ethoxy]ethoxy}ethyl 4-methylbenzenesulfonate

Another preferred compound of Formula II is:

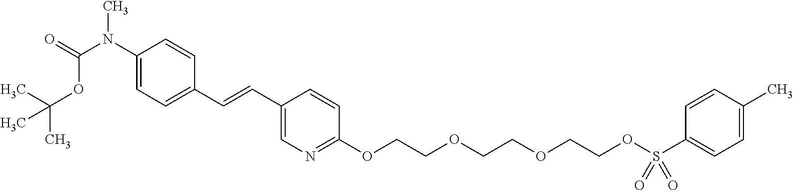 [SMILES: Cc1ccc(S(=O)(=O)OCCOCCOCCOc2ccc(/C=C/c3ccc(N(C)C(=O)OC(C)(C)C)cc3)cn2)cc1]

### 2-{2-[2-({5-[(E)-2-{4-[(tert-butoxycarbonyl)(methyl)amino]phenyl}vinyl]pyridin-2-yl}oxy)ethoxy]ethoxy}ethyl 4-methylbenzenesulfonate

Step 2 comprises a straight forward [F-18]fluoro labeling reaction from compounds of Formula II for obtaining compound of Formula I (if R=H) or compound of Formula III (if R=PG).

The radiolabeling method comprises the step of reacting a compound of Formula II with a F-18 fluorinating agent for obtaining a compound of Formula III or compound of Formula I. In a preferred embodiment, the [F-18]fluoride derivative is 4,7,13,16,21,24-Hexaoxa-1,10-diazabicyclo[8.8.8]-hexacosane K[F-18]F (Kryptofix K[F-18]F), K[F-18]F, H[F-18]F, KH[F-18]F₂, Cs[F-18]F, Na[F-18]F or tetraalkylammonium salt of [F-18]F (e.g. [F-18]tetrabutylammonium fluoride). More preferably, the fluorination agent is K[F-18]F, H[F-18]F, [F-18]tetrabutylammonium fluoride, Cs[F-18]F or KH[F-18]F₂, most preferably K[F-18], Cs[F-18]F or [F-18]tetrabutylammonium fluoride.

An even more preferred F-18 fluorinating agent is kryptofix/potassium[F-18]fluoride, preferably generated from [F-18]fluoride, kryptofix and potassium carbonate.

The radiofluorination reactions are carried out in acetonitrile, dimethylsulfoxide or dimethylformamide or a mixture thereof. But also other solvents can be used which are well known to someone skilled in the art. Water and/or alcohols can be involved in such a reaction as co-solvent. The radiofluorination reactions are conducted for less than 60 minutes. Preferred reaction times are less than 30 minutes. Further preferred reaction times are less than 15 min. This and other conditions for such radiofluorination are known to experts (Coenen, Fluorine-18 Labeling Methods: Features and Possibilities of Basic Reactions, (2006), in: Schubiger P. A., Friebe M., Lehmann L., (eds), PET-Chemistry—The Driving Force in Molecular Imaging. Springer, Berlin Heidelberg, pp. 15-50).

In one embodiment, 7.5-75 μmol, preferably 10-50 μmol, more preferably 10-30 μmol and even more preferably 12-25 μmol and even more preferably 13-25 μmol of compound of Formula II are used in Step 2.

In another embodiment, more than 7.5 μmol, preferably more than 10 μmol, and more preferable more than 12 μmol and even more preferably more than 13 μmol of compound of Formula II are used in Step 2.

In another embodiment, more than 5 mg, preferably more than 6 mg and more preferably more than 7 mg of compound of Formula II are used in Step 2.

In another embodiment 7 mg of compound of Formula II are used in Step 2.

In another embodiment 8 mg of compound of Formula II are used in Step 2.

In one preferred embodiment, the Radiofluorination of compound of Formula II is carried out in acetonitrile or in a mixture of acetonitrile and co-solvents, wherein the percentage of acetonitrile is at least 50%, more preferably at least 70%, even more preferably at least 90%.

Optionally, if R=PG, Step 3 comprises the deprotection of compound of Formula III to obtain compound of Formula I. Reaction conditions are known or obvious to someone skilled in the art, which are chosen from but not limited to those described in the textbook Greene and Wuts, Protecting groups in Organic Synthesis, third edition, page 494-653, included herewith by reference. Preferred reaction conditions are addition of an acid and stirring at 0° C.-180° C.; addition of an base and heating at 0° C.-180° C.; or a combination thereof.

Preferably the step 2 and step 3 are performed in the same reaction vessel.

Step 4 comprises the purification and optionally formulation of compound of Formula I using a HPLC separation system. Typically, the HPLC solvent eluents are acetonitrile/buffer mixtures. Preferably, a HPLC solvent eluent (e.g. mixtures of ethanol and aqueous buffers) is used, that can be part of the injectable Formulation of compound of Formula I. The collected product fraction can be diluted or mixed with other parts of the Formulation.

In a preferred embodiment, the HPLC solvent mixture is consisting of ethanol or an aqueous buffer or an ethanol/aqueous buffer mixture, wherein the aqueous buffer is consisting of components or excipient that can be injected into human. Examples for such aqueous buffer are solutions of sodium chloride, sodium phosphate buffer, ascorbic acid, ascorbate buffer or mixtures thereof.

In a preferred embodiment, the Method for manufacturing of compound of Formula I is carried out by use of a module (review: Krasikowa, Synthesis Modules and Automation in F-18 labeling (2006), in: Schubiger P. A., Friebe M., Lehmann L., (eds), PET-Chemistry—The Driving Force in Molecular Imaging. Springer, Berlin Heidelberg, pp. 289-316) which allows an automated synthesis. More preferably, the Method is carried out by use of an one-pot module. Even more preferable, the Method is carried out on commonly known non-cassette type modules (e.g. Ecker&Ziegler Modular-Lab, GE Tracerlab FX, Raytest SynChrom) and cassette type modules (e.g. GE Tracerlab MX, GE Fastlab, IBA Synthera, Eckert&Ziegler Modular-Lab PharmTracer), optionally, further equipment such as HPLC or dispensing devices are attached to the said modules.

In a second aspect the present invention is directed to a fully automated and/or remote controlled Method for production of compound of Formula I wherein compounds of Formula I, II and III and Steps 1, 2, 3 and 4 are described above. In a preferred embodiment this method is a fully automated process, compliant with GMP guidelines, that provides a Formulation of Formula I for the use of administration (injection) into human.

In a third aspect the present invention is directed to a Kit for the production of a pharmaceutical composition of compound of Formula I.

The Kit comprises a sealed vial containing kryptofix 2.2.2, potassium carbonate, an alcohol having a boiling point below 100° C. and water.

In one embodiment the Kit comprises a sealed vial containing a predetermined quantity of the compound of Formula II. Preferably, the Kit contains 1.5-75 μmol, preferably 7.5-50 μmol, more preferably 10-50 μmol and even more preferably 12-25 μmol and even more preferably 12-25 μmol and even more preferably 13-25 μmol of compound of Formula II.

In another embodiment the Kit contains more than 7.5 μmol, preferably more than 10 μmol and more preferably more than 12 μmol and even more preferably more than 13 μmol of compound of Formula II.

In another embodiment the Kit contains more than 5 mg, preferably more than 6 mg and more preferably more than 7 mg of compound of Formula II.

In another embodiment the Kit contains 7 mg of compound of Formula II.

In another embodiment the Kit contains 8 mg of compound of Formula II.

The kit also may contain a solvent or solvent mixture or the components for the solvent(mixture) for HPLC purification, such as acetonitrile/buffer mixtures or preferably solvents, solvent mixtures or components that are appropriate for the direct use for injection into patient.

Optionally, the Kit contains further components for manufacturing of compound of Formula I, such as solid-phase extraction cartridges, reagent for fluorination (as described above), acetonitrile or acetonitrile and a co-solvent, reagent for cleavage of deprotection group, solvent or solvent mixtures for purification, solvents and excipient for Formulation.

In one embodiment, the Kit contains a platform (e.g. cassette) for a “cassette-type module” (such as Tracerlab MX or IBA Synthera).

### DEFINITIONS

In the context of the present invention, preferred salts are pharmaceutically suitable salts of the compounds according to the invention. The invention also comprises salts which for their part are not suitable for pharmaceutical applications, but which can be used, for example, for isolating or purifying the compounds according to the invention.

Pharmaceutically suitable salts of the compounds according to the invention include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalene disulphonic acid, acetic acid, trifluoroacetic acid, propionic acid, lactic acid, tartaric acid, malic acid, citric acid, fumaric acid, maleic acid and benzoic acid.

Pharmaceutically suitable salts of the compounds according to the invention also include salts of customary bases, such as, by way of example and by way of preference, alkali metal salts (for example sodium salts and potassium salts), alkaline earth metal salts (for example calcium salts and magnesium salts) and ammonium salts, derived from ammonia or organic amines having 1 to 16 carbon atoms, such as, by way of example and by way of preference, ethylamine, diethylamine, triethylamine, ethyldiisopropylamine, monoethanolamine, diethanolamine, triethanolamine, dicyclohexylamine, dimethylaminoethanol, procaine, diben-zylamine, N methylmorpholine, arginine, lysine, ethylenediamine and N methylpiperidine.

The term Halogen or halo refers to Cl, Br, F or I.

The term “Amine-protecting group” as employed herein by itself or as part of another group is known or obvious to someone skilled in the art, which is chosen from but not limited to a class of protecting groups namely carbamates, amides, imides, N-alkyl amines, N-aryl amines, imines, enamines, boranes, N—P protecting groups, N-sulfenyl, N-sulfonyl and N-silyl, and which is chosen from but not limited to those described in the textbook Greene and Wuts, Protecting groups in Organic Synthesis, third edition, page 494-653, included herewith by reference. The amine-protecting group is preferably Carbobenzyloxy (Cbz), p-Methoxybenzyl carbonyl (Moz or MeOZ), tert-Butyloxycarbonyl (BOC), 9-Fluorenylmethyloxycarbonyl (FMOC), Benzyl (Bn), p-Methoxybenzyl (PMB), 3,4-Dimethoxybenzyl (DMPM), p-methoxyphenyl (PMP) or the protected amino group is a 1,3-dioxo-1,3-dihydro-2H-isoindol-2-yl (phthalimido) or an azido group.

The term “Leaving group” as employed herein by itself or as part of another group is known or obvious to someone skilled in the art, and means that an atom or group of atoms is detachable from a chemical substance by a nucleophilic agent. Examples are given e.g. in Synthesis (1982), p. 85-125, table 2 (p. 86; (the last entry of this table 2 needs to be corrected: “n-C₄F₉S(O)₂—O— nonaflat” instead of “n-C₄H₉S(O)₂—O— nonaflat”), Carey and Sundberg, Organische Synthese, (1995), page 279-281, table 5.8; or Netscher, Recent Res. Dev. Org. Chem., 2003, 7, 71-83, scheme 1, 2, 10 and 15 and others). (Coenen, Fluorine-18 Labeling Methods: Features and Possibilities of Basic Reactions, (2006), in: Schubiger P. A., Friebe M., Lehmann L., (eds), PET-Chemistry—The Driving Force in Molecular Imaging. Springer, Berlin Heidelberg, pp. 15-50, explicitly: scheme 4 pp. 25, scheme 5 pp 28, table 4 pp 30, FIG. 7 pp 33).

The term Sulfonyloxy refers to

—O—S(O)₂-Q wherein Q is optionally substituted aryl or optionally substituted alkyl.

The term “alkyl” as employed herein by itself or as part of another group refers to a C₁-C₁₀ straight chain or branched alkyl group such as, for example methyl, ethyl, propyl, isopropyl, butyl, isobutyl, tert-butyl, pentyl, isopentyl, neopentyl, heptyl, hexyl, decyl or adamantyl. Preferably, alkyl is C₁-C₆ straight chain or branched alkyl or C₇-C₁₀ straight chain or branched alkyl. Lower alkyl is a C₁-C₆ straight chain or branched alkyl.

The term “aryl” as employed herein by itself or as part of another group refers to monocyclic or bicyclic aromatic groups containing from 6 to 10 carbons in the ring portion, such as phenyl, naphthyl or tetrahydronaphthyl.

Whenever the term “substituted” is used, it is meant to indicate that one or more hydrogens on the atom indicated in the expression using “substituted” is/are replaced by one ore multiple moieties from the group comprising halogen, nitro, cyano, trifluoromethyl, alkyl and O-alkyl, provided that the regular valency of the respective atom is not exceeded, and that the substitution results in a chemically stable compound, i.e. a compound that is sufficiently robust to survive isolation to a useful degree of purity from a reaction mixture.

Unless otherwise specified, when referring to the compounds of Formula the present invention per se as well as to any pharmaceutical composition thereof the present invention includes all of the hydrates, salts, and complexes.

The term “F-18” means fluorine isotope ¹⁸F. The term“F-19” means fluorine isotope ¹⁹F.

### EXAMPLES

### Determination of Radiochemical and Chemical Purity

Radiochemical and chemical purities of 4-[(E)-2-(4-{2-[2-(2-[F-18]fluoroethoxy)ethoxy]-ethoxy}phenyl)vinyl]-N-methylaniline and 4-[(E)-2-(4-{2-[2-(2-[F-18]fluoroethoxy)ethoxy]-ethoxy}phenyl)vinyl]-N-methylaniline were determined by analytical HPLC (column: Atlantis T3; 150×4.6 mm, 3 μm, Waters; solvent A: 5 mM K₂HPO₄ pH 2.2; solvent B: acetonitrile; flow: 2 mL/min, gradient: 0:00 min 40% B, 0:00-05:50 min 40-90% B, 05:50-05:60 min 90-40% B, 05:60-09:00 min 40% B).
Retention time of 4-[(E)-2-(4-{2-[2-(2-[F-18]fluoroethoxy)ethoxy]-ethoxy}phenyl)-vinyl]-N-methylaniline: 3.5-3.9 min depending on the HPLC system used for quality control. Due to different equipment (e.g tubing) a difference in retention time is observed between the different HPLC systems. The identity of 4-[(E)-2-(4-{2-[2-(2-[F-18]fluoroethoxy)ethoxy]-ethoxy}phenyl)vinyl]-N-methylaniline was proofed by co-injection with the non-radioactive reference 4-[(E)-2-(4-{2-[2-(2-[F-19]fluoroethoxy)ethoxy]-ethoxy}phenyl)vinyl]-N-methylaniline.
Retention time of 4-[(E)-2-(6-{2-[2-(2-[F-18]fluoroethoxy)ethoxy]-ethoxy}pyridin-3-yl)vinyl]-N-methylaniline: 3.47 min. The identity of 4-[(E)-2-(6-{2-[2-(2-[F-18]fluoroethoxy)ethoxy]-ethoxy}pyridin-3-yl)vinyl]-N-methylaniline was proofed by co-elution with the non-radioactive reference -[(E)-2-(6-{2-[2-(2-[F-19]fluoroethoxy)ethoxy]-ethoxy}pyridin-3-yl)vinyl]-N-methylaniline.

### Preparation of 4-[(E)-2-(4-{2-[2-(2-[F-18]fluoroethoxy)ethoxy]-ethoxy}phenyl)vinyl]-N-methylaniline (Crude Product)

### Radiosynthesis on Tracerlab MX Using a Low Boiling Alcohol as Solvent in the QMA Eluent Mixture—General Procedure of the Synthesis

The synthesis is performed on GE TracerLab MX synthesizer equipped with a customized cassette (FIG. 3) and an appropriately assembled Kit. The setup of the synthesis automate is summarized in Table 1. [F-18]Fluoride is trapped on a QMA cartridge (C1). The activity is eluted with the eluent mixture (from “R1”) into the reactor. The solvent is removed while heating under gentle nitrogen stream and vacuum. Drying is repeated after addition of acetonitrile (from “R2”). The solution of precursor (from “R3”) is added to the dried residue and the mixture is heated for 10 min at 120° C. Then 2 mL 1.5M HCl (from “R4”) are added and solution is heated for 5 min at 110° C.

The crude product mixture is diluted with 1.2 mL 2M NaOH and 0.8 mL ammonium formate (1M) from syringe “R5”. 1 mL acetonitrile (from “R2”) and 0.5 mL ethanol (from “R6”) are added separately to the mixture and then transferred to the right syringe of the GE TracerLab MX automate. The reaction mixture containing the crude product is transferred into a 10 ml vial.

TABLE 1

| | |
|---|---|
| Vial R1 | specified eluent mixture (Example 1-4) |
| Vial R2 | 8 mL acetonitrile |
| Vial R3 | 8 mg precursor in 1.8 mL acetonitrile |
| Vial R4 | 2 mL HCl 1.5M |
| Vial R6 | 8 mL ethanol |
| Syringe R5 | 1.2 mL NaOH 2.0M |
| | 800 μL ammonium formate 1M |
| Cartridge C1 | QMA light (waters) conditioned with |
| | potassium carbonate 0.5M |

### Example 1

### Preparation of 4-[(E)-2-(4-{2-[2-(2-[F-18]fluoroethoxy)ethoxy]-ethoxy}phenyl)vinyl]-N-methylaniline (Crude Product) According to the General Procedure for the Synthesis

### Radiosynthesis on Tracerlab MX Using Ethanol as Organic Solvent in the QMA Eluent Mixture

The preparation was performed according to the General Procedure for the Synthesis. The composition of the F-18 eluent mixture and the results are listed in Table 2.

TABLE 2

| | |
|---|---|
| Vial R1 | 22 mg kryptofix 2.2.2 |
| | 7 mg potassium carbonate |
| | 300 μL ethanol |
| | 300 μL water |
| Start activity of [F-18]fluoride | 60.1 GBq |
| Recovery | 39.1 GBq |
| Product radiochemical purity (RCP), crude | 96% |
| product | |
| Radiochemical yield (calculated from RCP | 62% (not corrected for decay) |
| and Recovery) | |
| Content of the Acetate Impurity (HPLC) | No evaluable peak |

### Example 2

### Preparation of 4-[(E)-2-(4-{2-[2-(2-[F-18]fluoroethoxy)ethoxy]-ethoxy}phenyl)vinyl]-N-methylaniline (Crude Product) According to the General Procedure for the Synthesis

### Radiosynthesis on Tracerlab MX Using 2-Propanol as Organic Solvent in the QMA Eluent Mixture

The preparation was performed according to the General Procedure for the Synthesis. The composition of the F-18 eluent mixture and the results are listed in Table 3.

TABLE 3

| | |
|---|---|
| Vial R1 | 22 mg kryptofix 2.2.2 |
| | 7 mg potassium carbonate |
| | 300 μL 2-propanol |
| | 300 μL water |
| Start activity of [F-18]fluoride | 45.1 GBq |
| Recovery | 28.3 GBq |
| Product radiochemical purity (RCP), crude | 93% |
| product | |
| Radiochemical yield (calculated from RCP | 58% (not corrected for decay) |
| and Recovery | |
| Content of the Acetate Impurity (HPLC) | No evaluable peak |

### Example 3

### Preparation of 4-[(E)-2-(4-{2-[2-(2-[F-18]fluoroethoxy)ethoxy]-ethoxy}phenyl)vinyl]-N-methylaniline (Crude Product) According to the General Procedure for the Synthesis

Radiosynthesis on Tracerlab MX Using the Conventional QMA Eluent Mixture after Storage of 1 Year at Room Temperature

The preparation was performed according to the General Procedure for the Synthesis. The composition of the F-18 eluent mixture and the results are listed in Table 4.

TABLE 4

| | |
|---|---|
| Vial R1 (storage at room temperature for 1 | 22 mg kryptofix 2.2.2 |
| year) | 7 mg potassium carbonate |
| | 300 μL acetonitrile |
| | 300 μL water |
| Start activity of [F-18]fluoride | 37.5 GBq |
| Recovery | 22.8 GBq |
| Product radio-purity (RCP), crude product | 95% |
| Radiochemical yield (calculated from RCP | 57% (not corrected for decay) |
| and Recovery | |
| Content of the Acetate Impurity (HPLC) | 21 μg/ml [6 ml crude product |
| | solution] |

### Example 4

### Preparation of 4-[(E)-2-(4-{2-[2-(2-[F-18]fluoroethoxy)ethoxy]-ethoxy}phenyl)vinyl]-N-methylaniline (Crude Product) According to the General Procedure for the Synthesis

Radiosynthesis on Tracerlab MX Using the Conventional QMA Eluent Mixture after Storage of 8 Days at 60° C.

The preparation was performed according to the General Procedure for the Synthesis. The composition of the F-18 eluent mixture and the results are listed in the Table 5.

TABLE 5

| | |
|---|---|
| Vial R1 (storage at 60° C. for 8 days) | 22 mg kryptofix 2.2.2 |
| | 7 mg potassium carbonate |
| | 300 μL acetonitrile |
| | 300 μL water |
| Start activity of [F-18]fluoride | 55.6 GBq |
| Recovery | 37.9 GBq |
| Product radio-purity (RCP), crude product | 21% |
| Radiochemical yield (calculated from RCP | 14% (not corrected for decay) |
| and Recovery | |
| Content of the Acetate Impurity (HPLC) | 44 μg/ml [6 ml crude product |
| | solution] |

### Synthesis of 4-[(E)-2-(4-{2-[2-(2-[F-18]fluoroethoxy)ethoxy]-ethoxy}phenyl)vinyl]-N-methylaniline Radiosynthesis on Tracerlab MX Using a Low Boiling Alcohol in the QMA Eluent Mixture and Eckert&Ziegler Purification Unit—General Procedure for Synthesis and Purification

The synthesis is performed on a GE TracerLab MX synthesizer equipped with a customized cassette (FIG. 3) and an appropriately assembled Kit. The purification is performed on an Eckert & Ziegler Purification Unit. The filling of the injection loop of the HPLC is controlled by a fluid detector of the Eckert&Ziegler Purification unit. The setup of both automates and the results are summarized in Table 6. [F-18]Fluoride is trapped on a QMA cartridge (C1). The activity is eluted with the eluent mixture (from “R1”) into the reactor. The solvent is removed while heating under gentle nitrogen stream and vacuum. Drying is repeated after addition of acetonitrile (from “R2”). The solution of precursor (from “R3”) is added to the dried residue and the mixture is heated for 10 min at 120° C. Then 2 mL 1.5M HCl (from “R4”) are added and solution is heated for 5 min at 110° C.

The crude product mixture is diluted with 1.2 mL 2M NaOH and 0.8 mL ammonium formate (1M) from syringe “R5”. 1 mL acetonitrile (from “R2”) and 0.5 mL ethanol (from “R6”) are added separately to the mixture and then transferred to the right syringe of the GE TracerLab MX automate.

The mixture is transferred to the 10 mL sample injection loop of the semi-preparative HPLC using the right syringe of the GE TracerLab MX automate via a liquid sensor which controlls the end of the loading. The mixture is loaded to the semi-preparative HPLC column (Synergi Hydro-RP, 250×10 mm, Phenomenex). A mixture of 60% ethanol and 40% ascorbate buffer (4.32 g/l sodium ascorbate and 0.66 g/l ascorbic acid) is flushed through the column with 6 mL/min. The product fraction is collected directly during 60 sec into the product vial Formulation basis (consisting of 4 ml PEG400 and 16 ml water).

TABLE 6

| | |
|---|---|
| Vial R1 | specified eluent mixture (Example 5-7) |
| Vial R2 | 8 mL acetonitrile |
| Vial R3 | 8 mg precursor in 1.8 mL acetonitrile |
| Vial R4 | 2 mL HCl 1.5M |
| Vial R6 | 8 mL ethanol |
| Syringe R5 | 1.2 mL NaOH 2.0M |
| | 800 μL ammonium formate 1M |
| Cartridge C1 | QMA light (waters) conditioned with potassium |
| | carbonate 0.5M |
| HPLC column | Synergi Hydro-RP, 250* × 10 mm, 10 μm 80 Å, |
| | Phenomenex |
| HPLC solvent | 60% ethanol, 40% ascorbate buffer |
| | (4.3 g/l sodium ascorbate and 0.7 g/l ascorbic acid) |
| HPLC flow | 6 mL/min |

### Example 5

### Preparation of 4-[(E)-2-(4-{2-[2-(2-[F-18]fluoroethoxy)ethoxy]-ethoxy}phenyl)vinyl]-N-methylaniline According to the General Procedure for the Synthesis and Purification

### Radiosynthesis on Tracerlab MX Using Ethanol as Organic Solvent in the QMA Eluent Mixture and Eckert&Ziegler Purification Unit

The preparation was performed according to the General Procedure for the Synthesis and purification. The composition of the F-18 eluent mixture and the results are listed in Table 7.

TABLE 7

| | |
|---|---|
| Vial R1 | 22 mg kryptofix 2.2.2 |
| | 7 mg potassium carbonate |
| | 300 μL ethanol |
| | 300 μL water |
| Start activity of [F-18]fluoride | 44.8 GBq |
| Product activity | 17.5 GBq |
| Product radiochemical purity (RCP) | 99% |
| Radiochemical yield | 39% (not corrected for decay) |
| Content of the Acetate Impurity (HPLC) | 0.11 μg/ml [16 ml product |
| | solution] |

### Example 6

### Preparation of 4-[(E)-2-(4-{2-[2-(2-[F-18]fluoroethoxy)ethoxy]-ethoxy}phenyl)vinyl]-N-methylaniline According to the General Procedure for the Synthesis and Purification

Radiosynthesis on Tracerlab MX Using 2-Propanol as Organic Solvent in the QMA eluent mixture and Eckert&Ziegler Purification Unit

The preparation was performed according to the General Procedure for the Synthesis and purification. The composition of the F-18 eluent mixture and the results are listed in the Table 8.

TABLE 8

| | |
|---|---|
| Vial R1 | 22 mg kryptofix 2.2.2 |
| | 7 mg potassium carbonate |
| | 300 μL 2-propanol |
| | 300 μL water |
| Start activity of [F-18]fluoride | 74.4 GBq |
| Product activity | 27.4 GBq |
| Product radiochemical purity (RCP) | 99% |
| Radiochemical yield | 37% (not corrected for decay) |
| Content of the Acetate Impurity (HPLC) | 0.12 μg/ml [16 ml product |
| | solution] |

### Example 7

### Preparation of 4-[(E)-2-(4-{2-[2-(2-[F-18]fluoroethoxy)ethoxy]-ethoxy}phenyl)vinyl]-N-methylaniline According to the General Procedure for the Synthesis and Purification

Radiosynthesis on Tracerlab MX Using the Conventional QMA Eluent Mixture after Storage of 1 Year at Room Temperature and Eckert&Ziegler Purification Unit

The preparation was performed according to the General Procedure for the Synthesis and purification. The composition of the F-18 eluent mixture and the results are listed in Table 9.

TABLE 9

| | |
|---|---|
| Vial R1 (storage at room temperature for 1 | 22 mg kryptofix 2.2.2 |
| year) | 7 mg potassium carbonate |
| | 300 μL acetonitrile |
| | 300 μL water |
| Start activity of [F-18]fluoride | 35.8 GBq |
| Product activity | 11.8 GBq |
| Product radiochemical purity (RCP) | 99% |
| Radiochemical yield | 33% (not corrected for decay) |
| Content of the Acetate Impurity (HPLC) | 0.95 μg/ml [16 ml product |
| | solution] |

### DESCRIPTION OF THE FIGURES

FIG. 1 Setup of Tracerlab FX_{N} for purification with re-Formulation (adopted from tracerlab software)

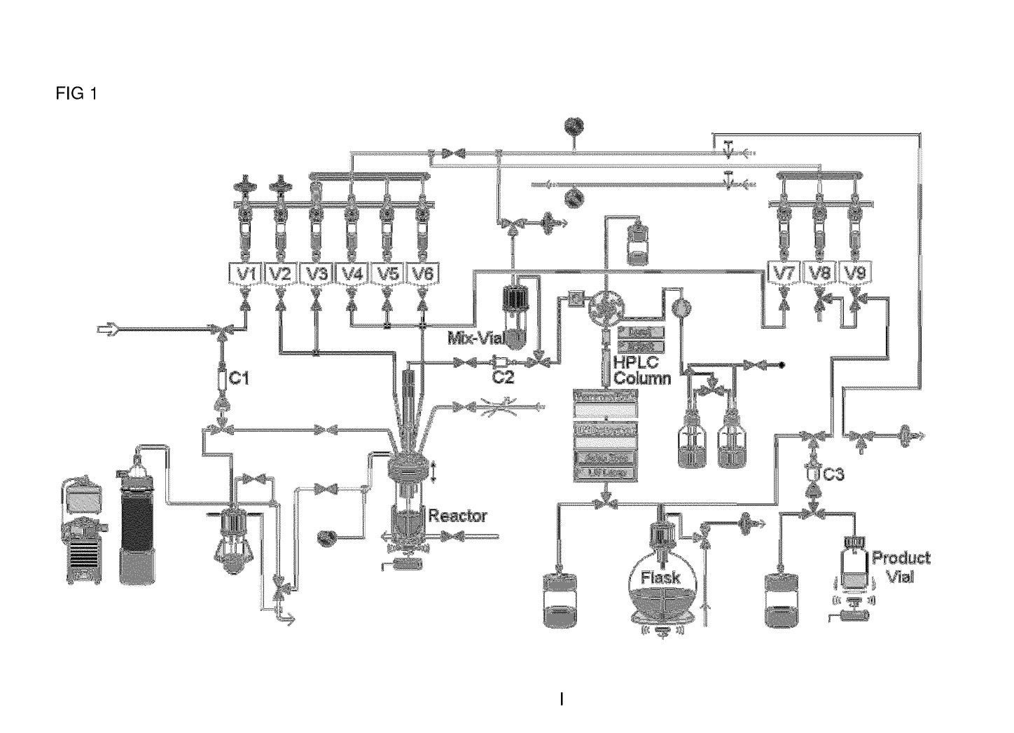

FIG. 2 Setup of Tracerlab FX_{N} for purification without re-Formulation (adopted from tracerlab software)

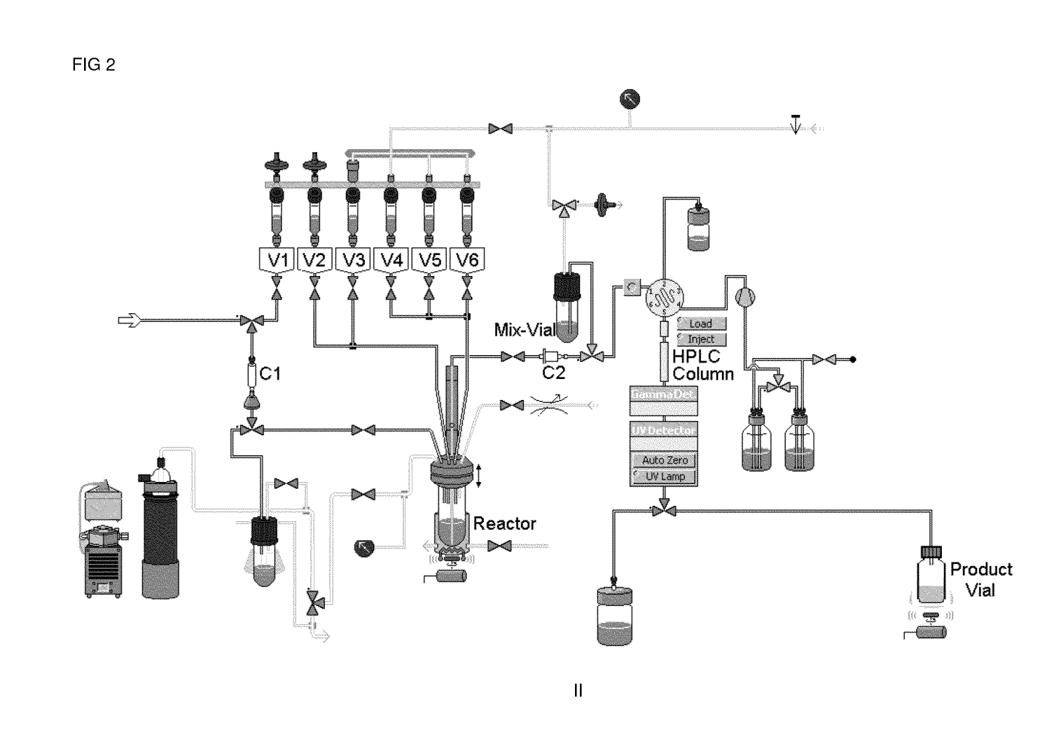

FIG. 3 Setup of Tracerlab MX (adopted from Coincidence FDG software)

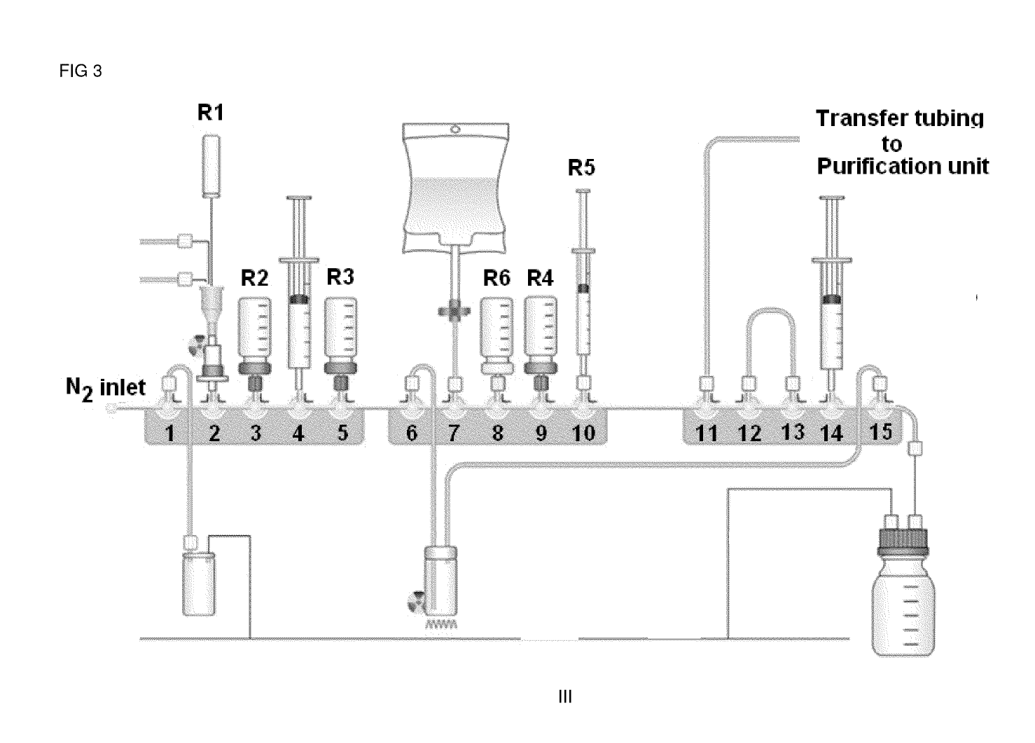

FIG. 4 Schematic illustration of process and equipment for manufacturing of F-18 labeled fluoropegylated (aryl/heteroaryl vinyl)-phenyl methyl amines comprising three parts: A) Synthesis, B) HPLC, C) Formulation; including (1) vials for reagents and solvents, (2) a reaction vessel, (3) target line for F-18, optionally gas lines, vacuum ect., (4) optionally fluid detector or filter ect., (5) injection valve, (6) HPLC column, (7) valve for peak cutting, (W) waste line(s), (8) vessel for collection/dilution of HPLC fraction, (9) solvent vials for washing and elution, (10) valve, (11) cartridge, e.g. C18 cartridge for trapping of the product, (12) valve.

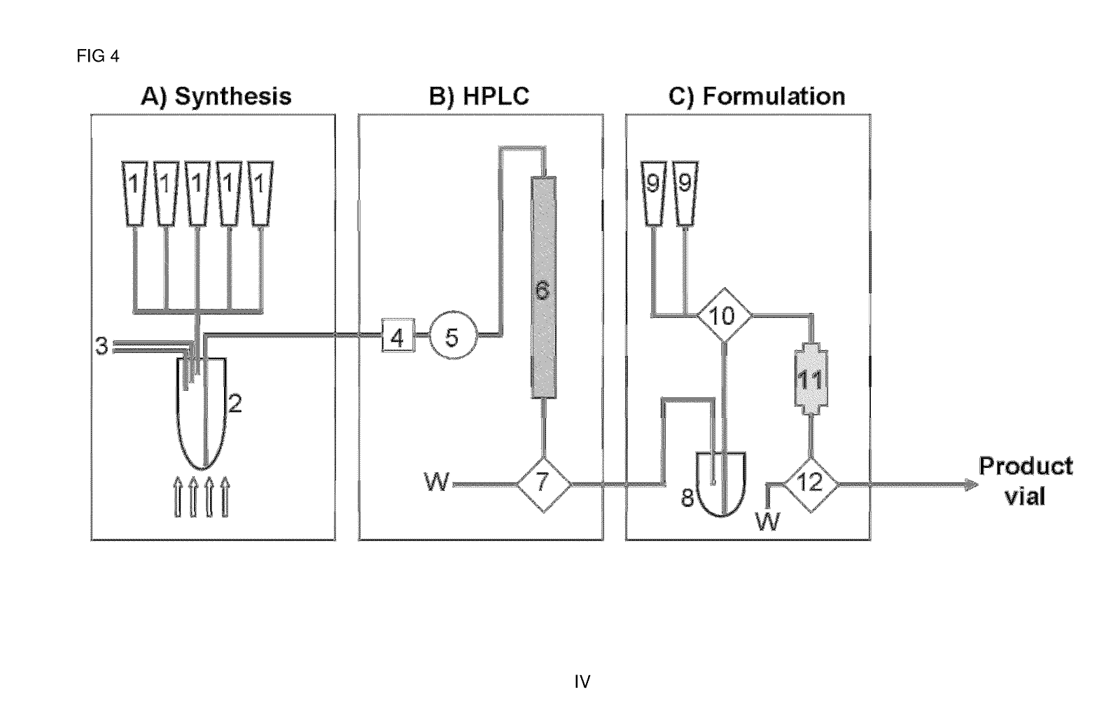

FIG. 5 Schematic illustration of process and equipment for manufacturing of F-18 labeled fluoropegylated (aryl/heteroaryl vinyl)-phenyl methyl amines comprising two parts: A) Synthesis, B) HPLC; including (1) vials for reagents and solvents, (2) a reaction vessel, (3) target line for F-18, optionally gas lines, vacuum ect., (4) optionally fluid detector or filter etc., (5) injection valve, (6) HPLC column, (7) valve for peak cutting.

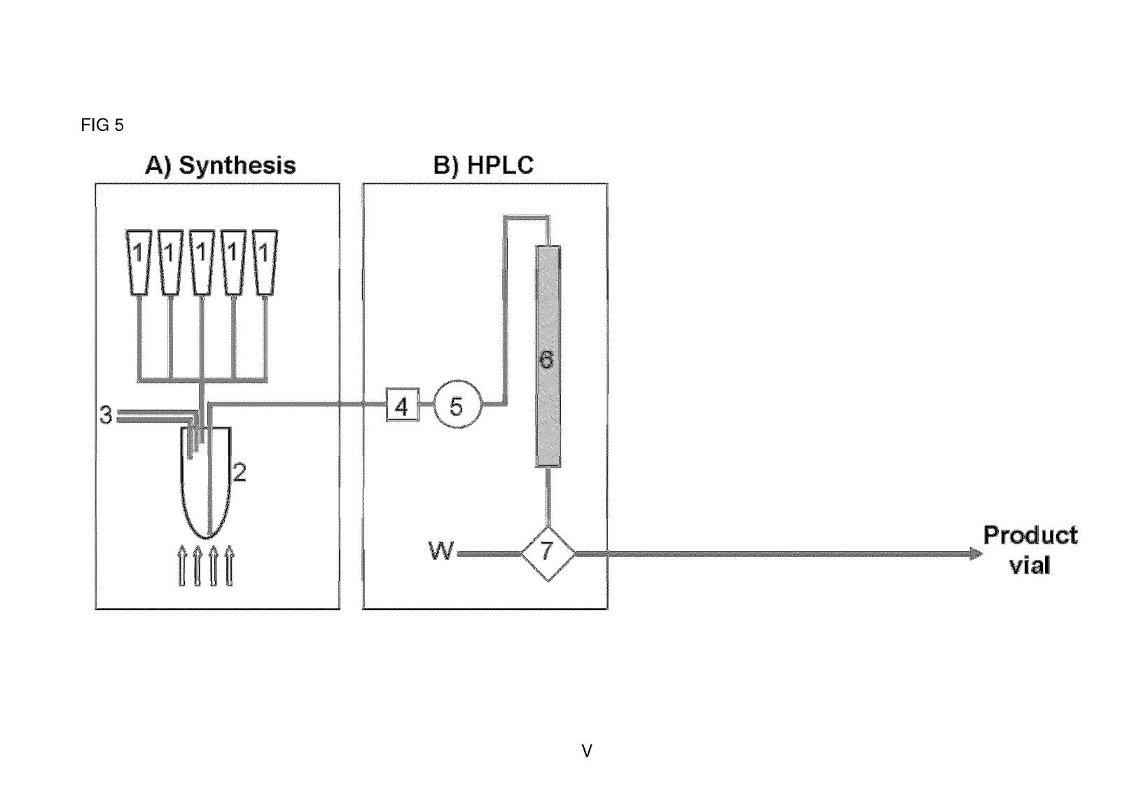

1. A Method for producing a compound of Formula I 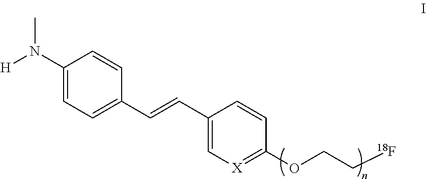
comprising the steps of:
Step 1: Preparation a fluorinating agent by eluting [18F]fluoride trapped on a cartridge using a mixture of cryptand, base, water and an alcohol having a boiling point below 100° C. followed by evaporation of the solvents,
Step 2: Radiolabeling compound of Formula II with the F-18 fluorinating agent, to obtain compound of Formula I, if R=H or to obtain compound of Formula III, if R=PG 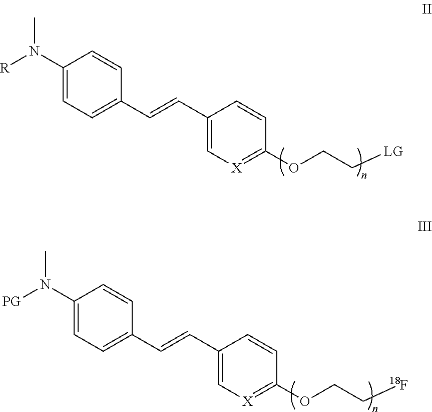
Step 3:If R=PG, cleavage of the protecting group PG to obtain a compound of Formula I
Step 4: Purification and optionally formulation of a compound of Formula I,
wherein:
n=1-6,
X is selected from the group consisting of
a) CH,
b) N,
R is selected from the group consisting of
a) H,
b) PG,
PG is an “Amine-protecting group”,
and
LG is a leaving group.

2. A method according to claim 1, wherein the alcohol is selected from the group consisting of ethanol or 2-propanol.

3. A method according to claim 2, wherein the alcohol is ethanol.

4. A method according to claim 1, wherein the [18F]fluoride is trapped on an anion exchange resin.

5. A method according to claim 4, wherein in step 1 a fluorinating agent is prepared by eluting [18F]fluoride trapped on a anion exchange resin using a mixture of kryptofix 2.2.2, potassium carbonate, water and a C2-C3 alcohol having a boiling point between 75° C. and 85° C. followed by evaporation of the solvents.

6. A method according to claim 1, wherein in step 1 a fluorination agent is prepared by eluting [18F]fluoride trapped on a QMA resin using a mixture of kryptofix 2.2.2, potassium carbonate, water and ethanol followed by evaporation of the solvents.

7. A method according to claim 1, wherein in step 1 a fluorination agent is prepared by eluting [18F]fluoride trapped on a QMA using a mixture of kryptofix 2.2.2, potassium carbonate, ethanol or 2-propanol and water followed by evaporation of the solvents.

8. A method according to claim 1, wherein in step 1 a fluorination agent is prepared by eluting [18F]fluoride trapped on a QMA using a mixture of kryptofix 2.2.2, potassium carbonate, ethanol and water followed by evaporation of the solvents.

9. A method according to claim 1, wherein PG is selected from the group consisting of:
a) Boc,
b) Trityl and
c) 4-Methoxytrityl.

10. A method according to claim 1, wherein LG is selected from the group consisting of:
a) Halogen and
b) Sulfonyloxy,
Halogen is chloro, bromo or iodo.

11. A method according to claim 10, wherein Sulfonyloxy is selected from the group comprising:
a) Methanesulfonyloxy,
b) p-Toluenesulfonyloxy,
c) (4-Nitrophenyl)sulfonyloxy,
d) (4-Bromophenyl)sulfonyloxy.

12. A method according to claim 1, wherein n=3 and X=CH.

13. A method according to claim 1, wherein n=3, X=CH, R=Boc, and LG=Methanesulfonyloxy.

14. A method according to claim 1, wherein the purification method in step 4 is an HPLC method.

15. A method according to claim 14, wherein the HPLC solvent is selected from the group consisting of ethanol, an aqueous buffer or an ethanol/aqueous buffer mixture.

16. A method according to claim 15, wherein the aqueous buffer is selected from the group of solutions of sodium chloride, sodium phosphate buffer, ascorbic acid, ascorbate buffer, or mixtures thereof.

17. A method according to claim 1, wherein the method is performed as a fully automated process.
